(19) United States
(12) Patent Application Publication
(10) Pub. No.: US 2005/0203627 A1
(43) Pub. Date: Sep. 15, 2005

(54) Intervertebral disc prosthesis

(75) Inventors: Munchi Soli Choksey, Fillongley (GB); Simon Nicholas Collins, Tetbury (GB); Sarah Catherine Taylor, Tetbury (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Corin Limited, Cirencester (GB)

(21) Appl. No.: 11/040,645

(22) Filed: Jan. 21, 2005

(30) Foreign Application Priority Data
Jan. 23, 2004 (GB) 0401489.0

(51) Int. Cl. A61F 2/44

(52) U.S. Cl. 623/17.15; 623/17.14

(57) ABSTRACT

An intervertebral prosthesis which is designed to be at least partially received within an intervertebral disc space. The intervertebral disc space is defined between an end plate of a first vertebral body and an adjacent end plate of a second, adjacent vertical body. The prosthesis comprises a first component which is designed to be attached to the first vertebral body and a second component which is designed to be attached to the second vertebral body. The first and second components comprise surfaces which, in use, are directed towards the opposing end plate and which extend substantially entirely across the part of the component which is received within the intervertebral disc space. The surfaces of both the first and second components define respective monotonic curves which have no discontinuities therein.

### FIELD OF THE INVENTION

The present invention relates to an intervertebral disc prosthesis.

### BACKGROUND OF THE INVENTION

A large number of people suffer from back pain and, in particular, in those regions of the neck and the back where the spinal column has a significant freedom of movement. Thus the neck and the lumber region are frequent sites of debilitating pain.

FIG. 1a shows two adjacent vertebrae 10a and 10b with an intervertebral disc 12 located in an intervertebral space 13 formed between the two vertebrae 10a and 10b.

Each vertebrae consist of a vertebral body 14, which is the anterior, more massive part of the bone that gives strength to the vertebral and supports body weight. A vertebral arch 16 is formed to the posterior of the vertebral body 14 and, as shown in FIG. 1b, is formed by right and left pedicles 18a and 18b and right and left lamina 20a and 20b. The pedicles 18a and 18b and lamina 20a and 20b also define a vertebral foramen 22, through which the spinal cord (not shown) passes.

Right and left superior articular processes 15a and 15b project upwardly from the junction of the right and left pedicles 18a and 18b with the right and left laminae 20a and 20b. At the same time right and left inferior articular processes, shown in FIG. 1b as 17, project downwardly from the same junction. The superior and inferior articular processes 15 and 17 of adjacent vertebrae 10a and 10b join to form the zygapophysical joint, also known as the facet joint, which permits gliding movements between the two adjacent vertebrae.

Surgery may be performed on the spine for a variety of reasons. These reasons may include when a trauma occurs to the neck or back which results in the rupture of one or more intervertebral discs. Other examples are when herniation or protrusion of an intervertebral disc occurs, for example due to degradation of the disc. If conservative treatment of back pain does not result in an improvement in the condition, or the damage to the disc is unlikely to heal using conservative methods, then spinal surgery may be used in an attempt to correct the problem, alleviate pain and/or maintain the spinal column in a form suitable for protecting the spinal cord.

Previous surgical methods employed to treat a degenerated, ruptured or fractured disc include the removal of a portion, or all of, the disc. Following the removal of the disc, the adjacent vertebrae may be fused using a variety of methods, for example rods screws, plates, bone grafts or fusion cages.

However, the use of spinal fusion affects the biomechanics of the spine and may cause other parts of the spine to degenerate more rapidly than anticipated or usual due to changes in the motion of, or loading applied to, other areas of the spine. For example, removal of an intervertebral disc from the intervertebral space between the C3-C4 vertebrae may accelerate deterioration of the C2-C3 or C4-C5 segments. One reason for this accelerated deterioration is that spinal fusion results in a loss of the rotational or translational movement between the fused vertebrae which is possible when a natural disc is present.

More recently alternative procedures have been proposed for corrective spinal surgery. These alternatives allow some degree of movement between the vertebrae after the removal of degenerated, ruptured or fractured discs.

One procedure is disclosed in U.S. Pat. No. 5,865,846 where an intervertebral disc prosthesis is used which has relatively stiff concaval/convex elements at least partially surrounding a relatively supple central nucleus portion. Information is obtained regarding the size, shape and nature of the patient's damaged spine using methods such as CT or MRI scans. Once the information has been collated then one or more prosthetic disc units are constructed and implanted into the patient's spine.

Another procedure is disclosed in WO 00/23015. Here an intervertebral disc prosthesis is described which comprises two components; a ball component which is attached to one vertebrae and a trough component which is attached to an adjacent vertebrae. The trough component includes a generally concave surface with a flat portion running therethrough. When the ball component and the trough component are inserted into the prepared intervertebral space, and are attached to the respective adjacent vertebrae, they inter-engage to permit a degree of rotational and translational motion of the vertebrae.

Although these intervertebral prostheses allow for some rotational motion and, in the case of WO 00/23015 some translational motion, this motion may be restricted, thereby affecting the biomechanics of the spinal column. Once again this may lead to excessive stresses on other parts of the spine and/or on certain areas of the prosthesis, thereby causing enhanced wear.

It is therefore desired to provide an intervertebral disc prosthesis which provides enhanced rotational and translational movement.

### SUMMARY OF THE INVENTION

According to the present invention there is provided an intervertebral prosthesis adapted to be at least partially received within an intervertebral disc space defined between a first endplate of a first vertebral body and a second endplate of a second, adjacent vertebral body, said prosthesis comprising; a first component adapted to be attached to said first vertebral body, said first component comprising a first surface which, in use, is directed towards said second endplate and extends substantially entirely across that part of the first component received within the intervertebral disc space; and a second component adapted to be attached to said second vertebral body, said second component comprising a second surface engageable with said first surface to permit articulation of said prosthesis, said second surface, in use, being directed towards said first endplate and extending substantially entirely across that part of the second component received within the intervertebral disc space, both said first and second surfaces defining respective monotonic curves having no discontinuities therein.

Advantageously, the first surface is defined by a concave articulating surface and said second surface is defined by a convex articulating surface.

More advantageously, the first and second surfaces are defined by a respective single centre and radius of curvature.

Most advantageously, the first and second surfaces are defined by the same single centre of curvature.

Preferably, the centre of curvature is posterior of a coronal plane through the centre of the first and second vertebral bodies.

Preferably, the centre of curvature lies on a median plane through the centre of the first and second vertebral bodies.

Advantageously, at least one said first and second surfaces is provided with one or more apertures for the receipt of attachment means with which to secure the component to the respective vertebral body.

Preferably, the apertures are disposed anterior to a coronal plane through the centre of the first and second vertebral bodies.

Preferably, the apertures are countersunk such that said attachment means does not protrude therefrom.

Advantageously, at least one of the first and second components further comprises an attachment surface adapted to be attached to an anterior surface of said first or second vertebral body.

Advantageously, the first component further comprises a first attachment surface adapted to be attached to an anterior surface of said first vertebral body, said attachment surface having a first configuration; and the second component further comprises a second attachment surface adapted to be attached to an anterior surface of said second vertebral body, said attachment surface having a second configuration, the first attachment surface and the second attachment surface being configured such that said first attachment surface will nest with said second attachment surface. Providing an intervertebral prosthesis with an attachment surface adapted to nest with the attachment surface of an adjacent intervertebral prosthesis is advantageous since nesting the attachment surfaces optimises use of the limited bone surface available, thereby giving optimal attachment characteristics and allowing for multi-level disc replacements.

It is also preferable that one of said first component or said second component is joined by an intermediate portion to a third component having a further surface; said intermediate portion being designed such that said first or second portion and said third portion are sufficiently spaced to be at least partially received within the adjacent intervertebral disc spaces.

Preferably, the first attachment surface is provided with one or more apertures for the receipt of attachment means with which to secure said first component to said first vertebral body and said second attachment surface is provided with one or more apertures for the receipt of attachment means with which to secure said second component to said second vertebral body, said one or more apertures provided in said first attachment surface being spaced from a median plane passing through the centre of the prosthesis by a different amount from said one or more apertures provided in said second attachment surface. This facilitates nesting of the attachment surfaces. However, a further advantage of situating the apertures in different sagittal planes is that, should the prosthesis require replacement in a revision operation, the attachment surfaces of the revision prosthesis could be provided with apertures lying in different planes to those of the prosthesis being replaced. This would mean that the revision prosthesis could be secured into undamaged bone, i.e. bone not previously used for attachment, to provide a more secure attachment.

Preferably, the apertures provided in both said first and second attachment means are disposed symmetrically with respect to said median plane.

Advantageously, the first and second components further comprise attachment means for attachment to the first and second vertebral bodies.

Preferably, the attachment means is selecting from the group consisting of pegs, plates, posts and screws.

Alternatively or additionally the attachment means includes a surface coating to facilitate bone ingrowth. Preferably, the surface coating is selected from the list comprising Titanium or Cobalt Chrome plasma spray and/or an osteoconductive material such as hydroxyapatite.

Advantageously, the first and second components are formed of biocompatible material.

Preferably, the biocompatible material is selected from the list comprising of Titanium alloy, Cobalt Chrome Stainless Steel, Alumina, Zirconia or any combinations of metal or ceramic biomaterials. Advantageously, at least one of said first and second surfaces is provided with a coating or modified surface layer to enhance lubrication and wear properties. For example Titanium Nitride.

According to another arrangement there is provided an intervertebral prosthesis adapted to be partially received within an intervertebral disc space defined between a first endplate of a first vertebral body and a second endplate of a second, adjacent vertebral body, said prosthesis comprising; a first component having a first articulating surface which, in use, is received within the intermediate disc space and a first attachment surface having a first configuration adapted to be attached to an anterior surface of said first vertebral body; and a second component comprising a second articulating surface engageable with said first articulating surface and a second attachment surface having a second configuration adapted to be attached to an anterior surface of said second vertebral body; said first attachment surface and said second attachment surface being configured such that said first attachment surface will nest with said second attachment surface.

Advantageously, the first attachment surfaces is provided with one or more apertures for the receipt of attachment means with which to secure said first component to said first vertebral body and said second attachment surface is provided with one or more apertures for the receipt of attachment means with which to secure said second component to said second vertebral body, said one or more apertures provided in said first attachment surface being spaced from a median plane passing through the centre of the prosthesis by a different amount from said one or more apertures provided in said second attachment surface.

Preferably, the apertures provided in both said first and second attachment means are disposed symmetrically with respect to said median plane.

According to a further arrangement there is provided a one piece intervertebral prosthesis adapted for use in two adjacent intervertebral disc spaces, each of said disc spaces being defined between end plates of two vertebral bodies, said prosthesis comprising; a first portion having a surface which, in use, is at least partially received within one of said intervertebral disc spaces and defines a first articulating surface; a second portion having a surface which, in use, is at least partially received within the other of said intervertebral disc spaces and defines a second articulating surface; and an intermediate portion joining said first and second portions and sized such that said first and second portions are sufficiently spaced to be at least partially received within said adjacent intervertebral disc spaces. One advantage of using a one piece intervertebral prosthesis when replacing two adjacent discs is the fact it may reduce the time for a disc replacement operation, thereby reducing the time a patient needs to be under general anaesthetic. Furthermore, the use of a one piece prosthesis may allow attachment to be achieved using fewer screws, thereby minimising trauma to the vertebral body whilst providing a secure attachment. The use of a one piece intervertebral prosthesis may also provide greater strength to the vertebral body.

### BRIEF DESCRIPTION OF THE DRAWINGS

The preferred embodiments of the invention will now be described, by way of example, with reference to the accompanying drawings, in which:

FIG. 1a shows a side view of two adjacent vertebrae within an intervertebral disc therein between;

FIG. 1b shows a plan view of a vertebrae;

FIG. 2a shows a front view partially in cross-section of a first portion of an intervertebral disc prosthesis in accordance with a preferred embodiment of the present invention;

FIG. 2b shows a side view partially in cross-section of the first component of FIG. 2a;

FIG. 2c shows a perspective view of the first component of FIGS. 2a and 2b;

FIG. 3a shows a front view of a second component of an intervertebral disc prosthesis for use with the first component shown in FIGS. 2a-c;

FIG. 3b shows a perspective view of the second component of FIG. 3a;

FIG. 3c shows a side view partially in cross-section of the second component of FIGS. 3a and 3b;

FIG. 4 shows a front view of two nested intervertebral disc prostheses comprising first and second components as shown in FIGS. 2a-c and 3a-c;

FIG. 5a shows a posterior perspective view of a further design of an intervertebral prosthesis;

FIG. 5b shows an anterior perspective view of the intervertebral prosthesis shown in FIG. 5a;

FIG. 5c shows a posterior view of the intervertebral prosthesis shown in FIGS. 5a and 5b;

FIG. 6a shows a side view of the intervertebral prosthesis shown in FIGS. 5a-c;

FIG. 6b shows the intervertebral prosthesis of FIG. 6a in flexion;

FIG. 6c shows the intervertebral prosthesis of FIG. 6a in extension;

FIG. 7 shows an intervertebral prosthesis further comprising attachment keels;

FIG. 8 shows a side view partially in cross-section of an intervertebral disc prosthesis comprising two components in accordance with a further embodiment of the present invention;

FIG. 9 shows a plan view of one component of the intervertebral disc prosthesis shown in FIG. 8 attached to a vertebral body; and

FIGS. 10a-c show a side view of an intervertebral disc prosthesis according to a further embodiment of the present invention.

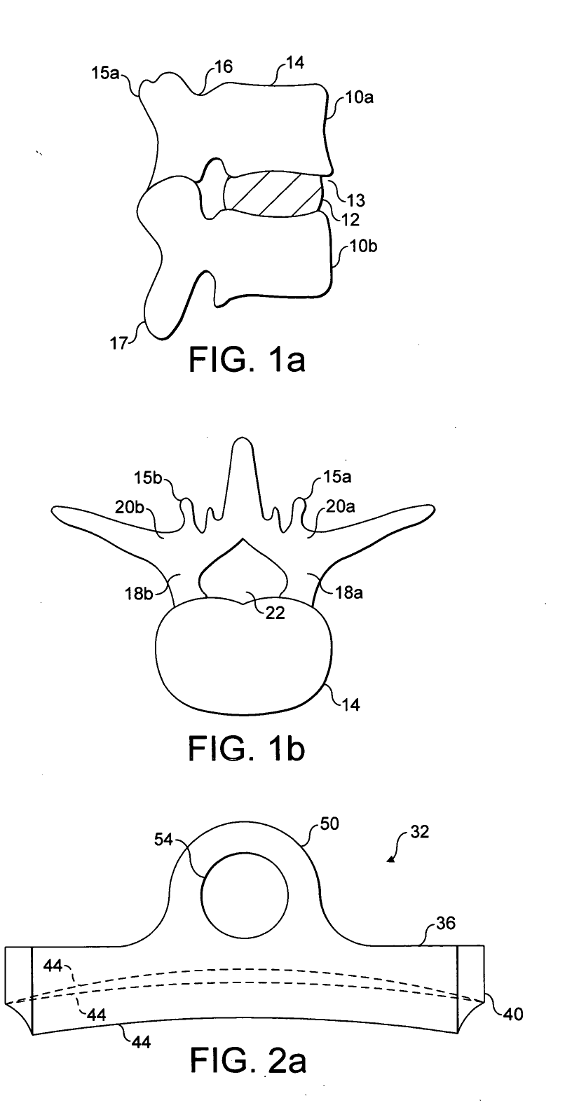

### DETAILED DESCRIPTION OF THE DRAWINGS

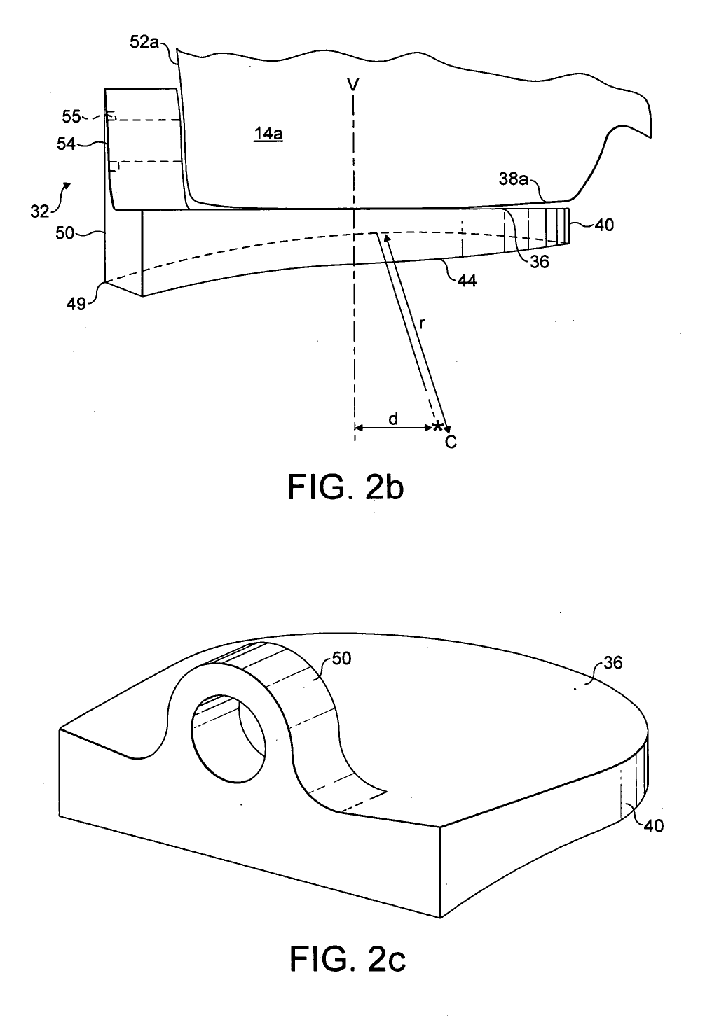

Referring to FIGS. 2-4 there is shown a first embodiment of an intervertebral disc prosthesis 30 according to the present invention. Prosthesis 30 includes a first prosthetic component 32 and a second prosthetic component 34, which are adapted to be received within at least part of an intervertebral disc space formed between two adjacent vertebral bodies, described subsequently as the superior and inferior vertebral bodies 14a and 14b, the vertebral bodies having vertebral endplates 38a and 38b. The first and second portions 32 and 34 interengage, as shown in FIG. 4, to form the prosthesis 30.

FIGS. 2a-c show the first prosthetic component 32 in more detail. The first prosthetic component 32 comprises a vertebrae engaging surface 36 which is configured to be attached to, or to lie in contact with, the prepared endplate 38a of the superior vertebral body 14a. The vertebrae engaging surface 36 terminates at a posterior edge in a downwardly depending wall 40. The wall 40 in turn merges with a uniformly concave articulating surface 44 defined by a part spherical surface having a single centre of curvature C and a single radius of curvature r, the centre of curvature having a displacement d from a coronal plane v through the centre of the vertebral endplates 38a and 38b. At an anterior edge 49 a flange 50 extends substantially vertically upwardly from the concave articulating surface 44 and is configured to lie in close proximity with an anterior surface 52a of the superior vertebral body 14a.

The flange 50 takes a male form. More specifically, in the embodiment shown in FIGS. 2-6, this form is a curved configuration sized to accommodate an aperture 54. The aperture 54 is sized to allow a fixation means, for example a bone screw, to pass through for attaching the flange 50 to the anterior surface 52a. The aperture 54 may further comprise a countersunk portion 55 to accept the head of the bone screw, thereby allowing the top of the screw to lie flush with a front surface 51 of the flange 50. In other words, the screw head will preferably lie completely below the surface of the flange (50). Advantageously a fixation means will be used in which the screw head is countersunk and in addition clips into recesses provided in the countersunk portion 55 (recesses not shown). An example of such a fixation means is the Cervive™ screw, details of which may be found in U.S. Pat. No. 6,613,053 issued on 9 Feb. 2003.

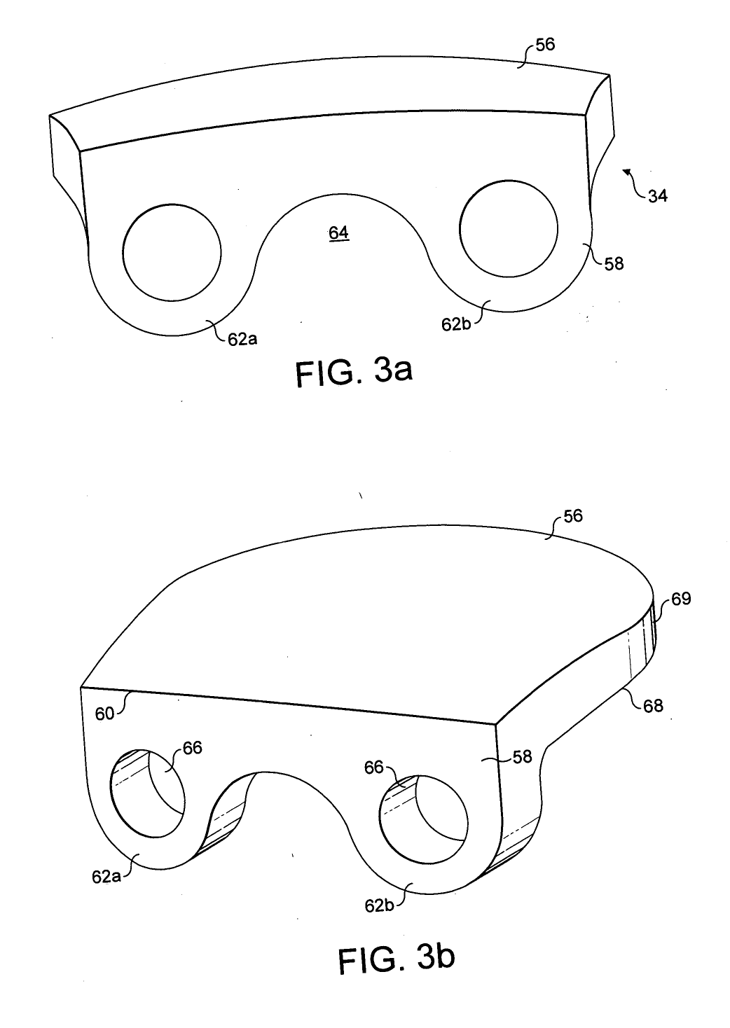

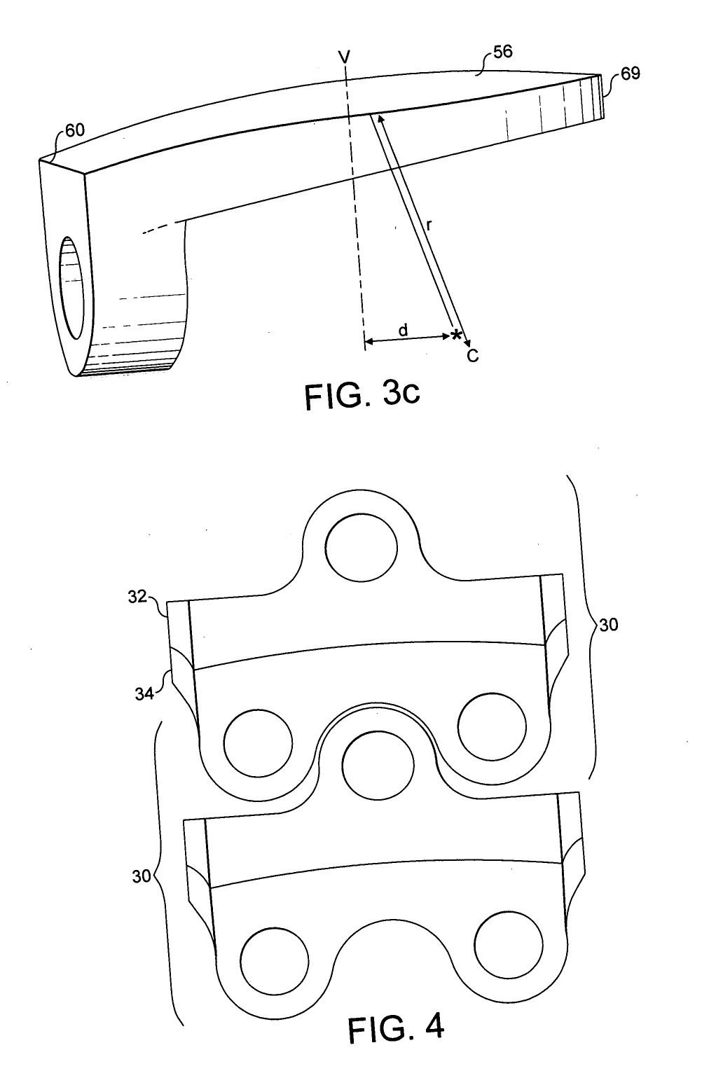

FIGS. 3a, 3b and 3c show the second prosthetic component 34 in more detail. The second prosthetic component 34 comprises a vertebrae engaging surface 68 which is configured to be attached to, or lie in contact with, the prepared endplate 38b of the inferior vertebral body 14b. The vertebrae engaging surface 68 terminates at a posterior edge in an upwardly extending wall 69. The wall 69 in turn merges with a uniformly convex articulating surface 56, defined by a part spherical surface designed to interengage with the concave articulating surface 44 to allow both rotational and translational motion. The convex articulating surface 56 has the same centre of curvature C, radius of curvature r and displacement d as the concave articulating surface 44. This configuration means that when the prosthesis 30 is located in the intervertebral space it will allow for flexion, extension, lateral bending and rotation of the spine in a manner which approximates as close as possible to normal movement.

The second prosthetic component 34 further comprises a flange 58 which depends substantially vertically downwardly from an anterior edge 60 of the convex articulating surface 56 and is configured to lie in close proximity with an anterior surface 52b of the inferior vertebrae 14b.

The flange 58 takes a female form complementary to that of the male flange 50. More specifically, in this embodiment the female flange 58 comprises two lugs 62a and 62b with a recess 64 formed between the lugs. The recess 64 is shaped to allow the male flange 50 to be at least partially received therein, as shown in FIG. 4. Each of the lugs 62a and 62b are sized to accommodate an aperture 66 sized to allow a fixation means, for example a bone screw, to pass through and attach the flange 58 to the anterior surface 52b. An example of a suitable bone screw is the Cervive™ screw previously disclosed. As with the aperture 54 of flange 50, the apertures 66 may be countersunk to accept the heads of the screws.

The concave and convex articulating surfaces 44 and 56 are preferably configured such that their centre of curvature C lies at a point in the median plane. The displacement d preferably lies in the range −3 mm (anterior of the coronal plane v) and +10 mm (posterior of the coronal plane v). Preferably the centre of curvature C is displaced towards the posterior (or rear) of the coronal plane v. Such a displacement allows for enhanced flexion and extension of the spine. Preferably the centre of curvature C lies within the subjacent vertebral body. Preferably the radius of curvature r lies in the range of 20 to 60 mm, more preferably 30 to 50 mm and still more preferably 35 to 45 mm.

The configuration of the flanges 50 and 58 as male and female forms means that should two adjacent intervertebral discs require replacement, either at the same time or during a revision operation, then the prosthesis 30 may be fitted with the flanges nesting rather than overlapping, i.e. with a portion of the flange of one component received within a recess formed in the flange of the adjacent component. Although the flanges 50 and 58 are shown having a curved form it will be understood that they may take other forms, for example with squared corners.

Additionally, although the first prosthetic component 32 is described as having a male flange 50 it will be understood that it is anticipated that the male and female flanges may be interchanged between the two prosthetic components. Thus, the first prosthetic component 32 may be provided with a female flange 58 and the second prosthetic component may be provided with a male flange 50.

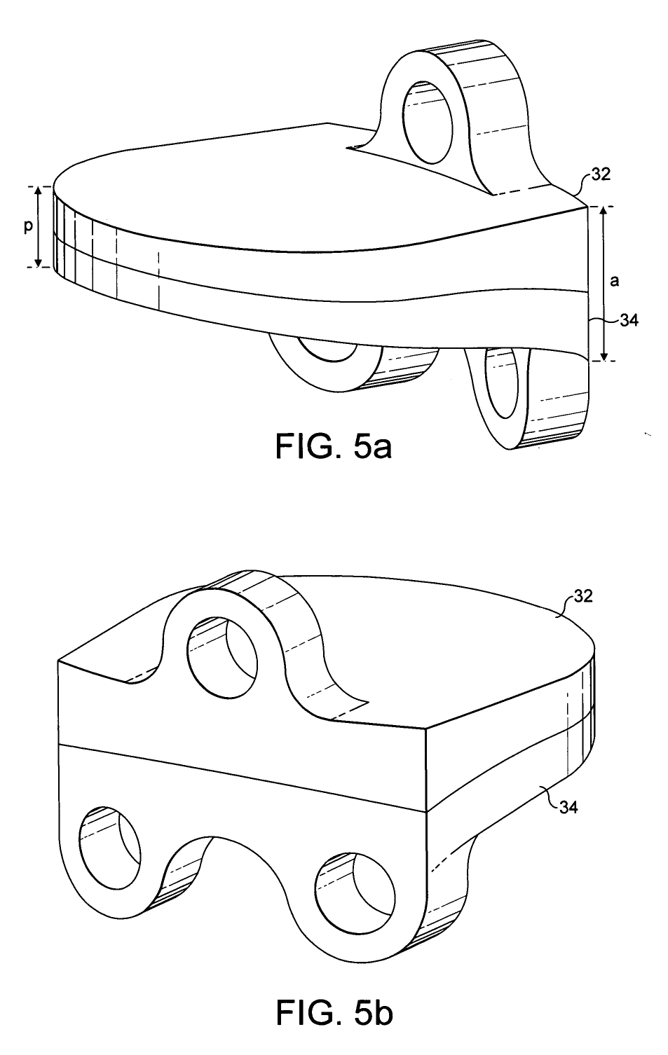

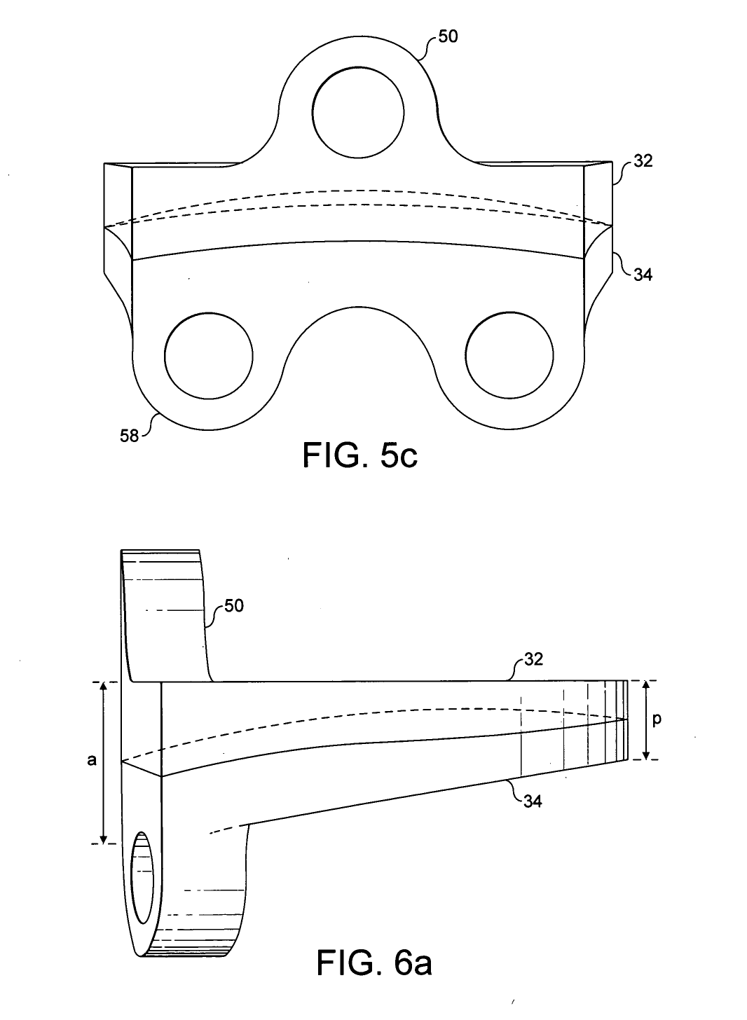

FIG. 5a-C show the intervertebral prosthesis 30 as assembled and in particular show the interaction between the concave and convex articulating surfaces 44 and 56. In order to allow lordosis of the spine the intervertebral disc prosthesis preferably occupies a space which is itself in the form of a trapezium, with an anterior dimension a being greater than posterior dimension p as shown in FIG. 5a.

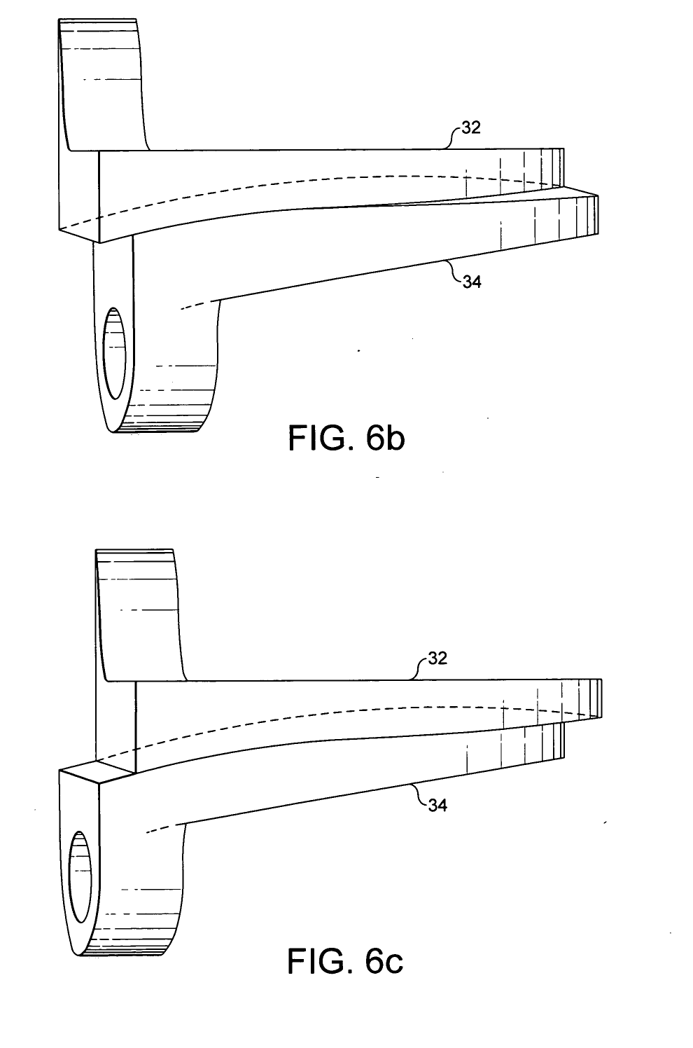

FIG. 6a shows a side view of the intervertebral prosthesis 30 and shows in more detail how the first and second prosthetic components 32 and 34 are designed to conform with the natural lordosis.

FIGS. 6b and 6c show the intervertebral prosthesis 30 in flexion and extension respectively.

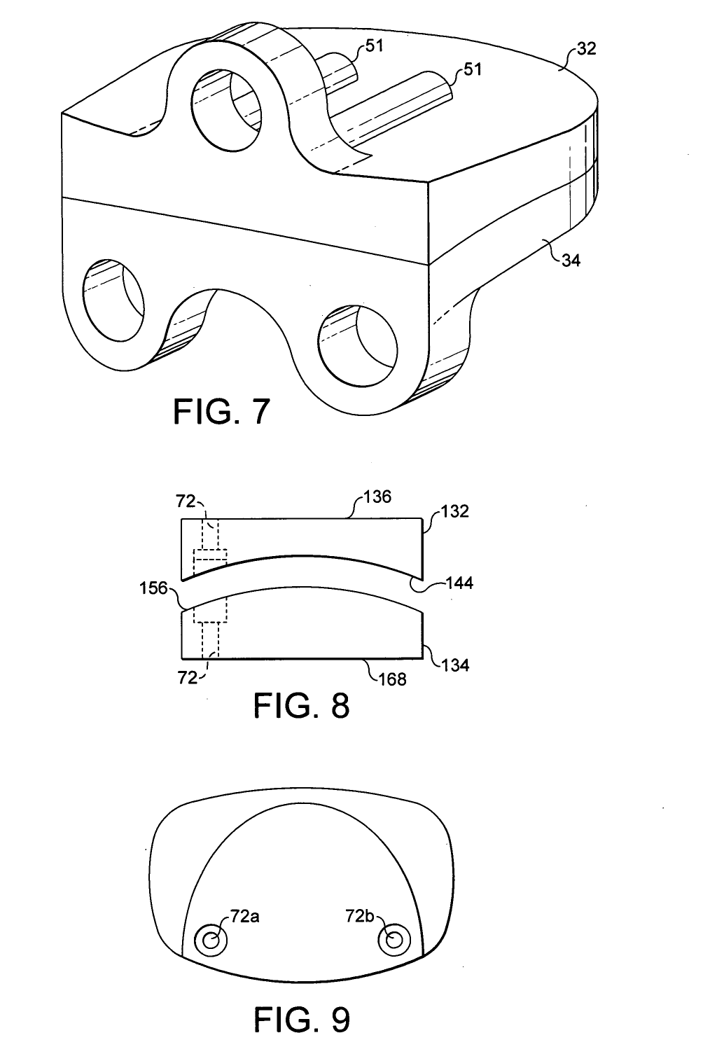

The attachment of the intervertebral prosthesis 30 may be enhanced by the use of one or more keels 51 provided on the vertebrae engaging surfaces 36 and 68 as is shown in FIG. 7. The use of such a keel 51 provides a greater surface area in contact with the bone and preferably the contact area of the prosthesis is provided with a surface coating to encourage bone ingrowth. In addition, the or each keel 51 will provide enhanced rotational and translational stability of the intervertebral prosthesis 30. Although the keel 51 is shown as being of semicircular cross-section it will be understood that other suitable cross-sections, for example rectangular or trapezoidal, may be used. Similarly, although the cross-section of each keel 51 is shown as being constant along the length of the keel 51, it will be understood that this is not a requirement, for example the keel 51 may be of increasing or decreasing cross-section along its length. Furthermore, although FIG. 7 shows two keels 51, one on either side of the median plane, it will be understood that only one keel may be provided, either on the median plane or offset to one side.

FIGS. 8 and 9 show a second embodiment according to the present invention, in which the flanges used to attach the prosthesis 30 to the vertebrae bodies 14a and 14b are omitted. For ease of reference the reference numerals for like components are the same as in the previously described embodiment, with a new stem numeral to indicate a different embodiment. Thus, as before, prosthesis 130 consists of a first prosthetic component 132 and a second prosthetic component 134. Similarly, the first prosthetic component 132 comprises a vertebrae engaging surface 136, which is configured to contact the prepared endplate 38a of a superior vertebrae 14a, and has a uniformly concave articulating surface 144. The concave articulating surface 144 is of part spherical form as described with regard to the previous embodiment.

The second prosthetic component 134 comprises a vertebrae engaging surface 168 adapted to lie in contact with a prepared end surface 38b of an inferior vertebrae 14b and has a uniformly convex articulating surface 156 of part spherical form as described with regard to the previous embodiment.

In place of the flanges 50 and 58, the first and second prosthetic components 132 and 134 comprise countersunk apertures 72a and 72b which are sized to receive bone screws, for example the Cervive Screw as previously discussed, which may be used to attach the prosthesis 130 to the vertebral bodies 14a and 14b. The apertures 72a and 72b are located anterior to a coronal plane through the centre of the vertebral bodies 14a and 14b to minimise their interference with the articulation between the concave and convex articulating surfaces 144 and 156. As before, the centre of curvature of the concave and convex articulating surfaces 144 and 156 is preferably located on the median plane to the posterior of the same coronal plane.

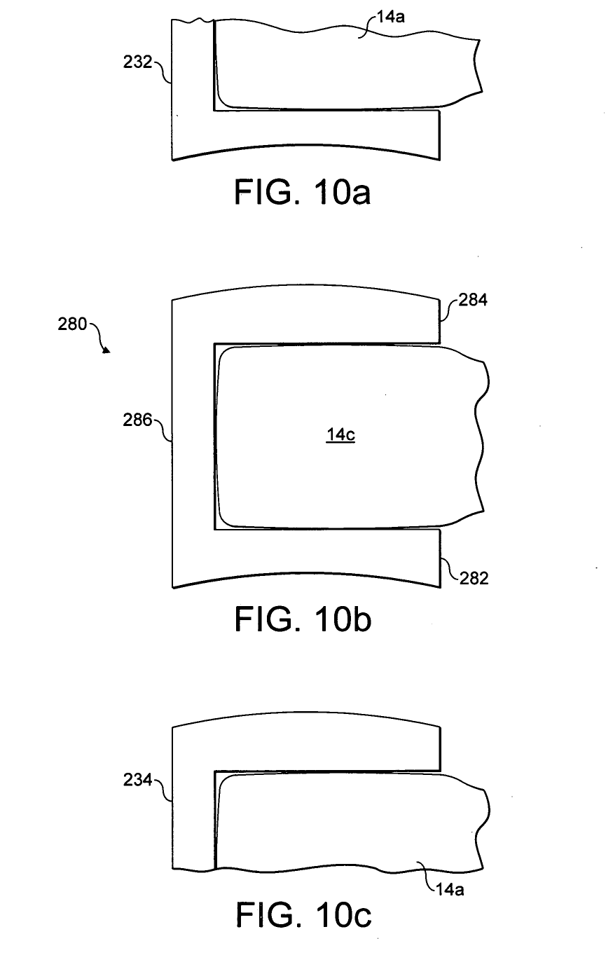

A third embodiment of the present invention is shown in FIGS. 10a-c. In this embodiment a prosthesis 230 is specifically designed for the replacement of two or more adjacent intervertebral discs. In the embodiment shown a three piece prosthesis 230 is provided, the prosthesis 230 comprising a one piece central prosthetic component 280 and two end prosthetic components 232 and 234. The end prosthetic components 232 and 234 correspond to the first prosthetic component 32 and second prosthetic component 34 previously described.

In one configuration the central prosthetic component 280 is configured to fit over the vertebrae intermediate the discs to be replaced, such that an upper prosthetic block 284 lies in the superior intervertebral space and a lower prosthetic block 282 lies in the inferior intervertebral space. The upper prosthetic block 284 is configured substantially similarly to the second prosthetic component 34 described in relation to FIGS. 3a-c and has a uniformly convex articulating surface 256 as its uppermost surface. At the same time the lower prosthetic block 282 is configured substantially similarly to the first prosthetic component 32 described in relation to FIGS. 2a-c and has a uniformly concave articulating surface 244 as its lowermost surface.

Rather than individual flanges being provided on each block, as in the first described embodiment, an intermediate portion 286 extends between the first and second prosthetic blocks 284 and 282 such that the upper prosthetic block 284 and lower prosthetic block 282 are held in spaced relation so as to be correctly located in the adjacent intervertebral spaces. Preferably the upper prosthetic block 284 has a vertebral engaging surface 268 and the second prosthetic block 282 has a vertebral engaging surface 236, these surfaces being connected by the intermediate portion 286 which comprises an anterior vertebral engaging surface 288.

Alternatively the central prosthetic component 280 may be configured to replace a damaged vertebrae and in so doing provide articulating surfaces on its upper and lower surfaces, 256 and 244, for interengagement with end prosthetic components 232 and 234.

The surgical procedure used to insert the intervertebral prosthesis is as follows. Firstly, information is found out about the size, shape and nature of the patient's damaged vertebral body, or bodies, using one or more of x-rays, CT scans and MRI scans. In particular, the anterior/posterior and lateral dimensions of the endplates of the vertebral bodies defining the relevant intervertebral disc space(s) are collated. In addition, the height of the relevant intervertebral disc space(s) are ascertained. A correctly sized intervertebral disc prosthesis is then chosen to correlate with the measured dimensions. The intervertebral disc or discs which require replacement are removed and the endplates of the respective vertebrae are then milled or otherwise prepared to receive the vertebral engaging surfaces of the prosthesis.

If a prosthesis is to be used according to the embodiments shown in FIGS. 2-4 and 6-7, i.e. with flanges, it may also be desirable to remove some of the anterior face of the vertebrae to allow the flanges to be sunk into the anterior face. This preparation may be by milling or any other suitable preparation method. However, it will also be understood that the flanges may sit directly on the anterior face of the vertebral body if desired, in particular if the remaining bone would not provide adequate fixation if part of the anterior face were removed.

The prosthesis is then offered up to the intervertebral space, preferably using a introducer tool. If bone screws are to be used as the primary method of fixation, then the introducer tool preferably includes apertures through which holes can be drilled, or which will allow access to pre-drilled holes, thereby facilitating correct placement of the screws into the vertebral body. The screws, may then be threaded into the vertebrae through the apertures to fix the components to the respective vertebrae. The screws can then be tightened to securely attach the prosthesis to the vertebral body.

Although the attachment of the prosthetic components 32, 34, 132, 134, 232, and 234 to the vertebrae has been described as by the use of bone screws it is understood that other methods of attachment may be used as either a primary or secondary method of attachment. For example, adhesives or staples may also be used. Other examples of fixation methods include the use of teeth, protrusions or webs to bite into the vertebrae to help securement. In addition, a coating may be provided on the vertebrae engaging surface(s) to encourage bone in-growth to provide additional fixation.

The intervertebral prosthesis is preferably made of a biocompatible material such as Cobalt Chrome or titanium. The articulating surfaces may be ceramic on ceramic or metal on metal or other suitable biocompatible materials. If desired the prosthesis may be coated, for example with Titanium Nitrite, to provide a more suitable attachment and/or bearing surface.

1. An intervertebral prosthesis adapted to be at least partially received within an intervertebral disc space defined between a first endplate of a first vertebral body and a second endplate of a second, adjacent, vertebral body, said prosthesis comprising:
a first component adapted to be attached to said first vertebral body, said first component comprising a first surface which, in use, is directed towards said second endplate and extends substantially entirely across that part of the first component received within the intervertebral disc space; and
a second component adapted to be attached to said second vertebral body, said second component comprising a second surface engageable with said first surface to permit articulation of said prosthesis, said second surface, in use, being directed towards said first endplate and extending substantially entirely across that part of the second component received within the intervertebral disc space, both said first and second surfaces defining respective monotonic curves having no discontinuities therein.

2. An intervertebral prosthesis as claimed in claim 1, wherein at least one of said first and second components further comprises an attachment surface adapted to be attached to an anterior surface of said first or second vertebral body.

3. An intervertebral prosthesis as claimed in claim 1, wherein
said first component further comprises a first attachment surface adapted to be attached to an anterior surface of said first vertebral body, said first attachment surface having a first configuration; and
said second component further comprises a second attachment surface adapted to be attached to an anterior surface of said second vertebral body, said second attachment surface having a second configuration,
said first attachment surface and said second attachment surface being configured such that said first attachment surface is capable of nesting with said second attachment surface.

4. An intervertebral prosthesis as claimed in claim 1, wherein one of said first component or said second component is joined by an intermediate portion to a third component having a further surface;
said intermediate portion being designed such that a first or second portion and said third portion are sufficiently spaced to be at least partially received within the adjacent intervertebral disc space.

5. An intervertebral prosthesis as claimed in claim 1, wherein one or both of said first or second components is provided with a keel.

6. An intervertebral prosthesis as claimed in claim 3, wherein said first attachment surface is provided with one or more apertures for the receipt of attachment means with which to secure said first component to said first vertebral body and said second attachment surface is provided with one or more apertures for the receipt of attachment means with which to secure said second component to said second vertebral body, said one or more apertures provided in said first attachment surface being spaced from a median plane passing through the centre of the prosthesis by a different amount from said one or more apertures provided in said second attachment surface.

7. An intervertebral prosthesis as claimed in claim 6, wherein the apertures provided in both said first and second attachment surfaces are disposed symmetrically with respect to said median plane.

8. An intervertebral prosthesis as claimed in claim 1, wherein at least one of said first and second surfaces is provided with one or more apertures for the receipt of attachment means with which to secure the respective component to the respective vertebral body.

9. An intervertebral prosthesis as claimed in claim 8, wherein said apertures are disposed anterior to a coronal plane through the centre of the first and second endplates.

10. An intervertebral prosthesis as claimed in claim 8, wherein the apertures are countersunk such that said attachment means does not protrude therefrom.

11. An intervertebral prosthesis as claimed in claim 1, wherein said first and second components further comprise attachment means for attachment to the first and second vertebral bodies.

12. An intervertebral prosthesis as claimed in claim 8, wherein said attachment means is selected from the group consisting of: pegs, posts, plates, and screws.

13. An intervertebral prosthesis as claimed in claim 11, wherein said attachment means includes a surface coating to facilitate bone ingrowth.

14. An intervertebral prosthesis as claimed in claim 8 further comprising a secondary attachment means.

15. An intervertebral prosthesis as claimed in claim 1, wherein said first surface is defined by a concave articulating surface and said second surface is defined by a convex articulating surface.

16. An intervertebral prosthesis as claimed in claim 1 wherein said first and second surfaces are defined by a respective single centre and radius of curvature.

17. An intervertebral prosthesis as claimed in claim 16, wherein said first and second surfaces are defined by the same single centre of curvature.

18. An intervertebral prosthesis as claimed in claim 17, wherein said centre of curvature is posterior of a coronal plane through the centre of first and second vertebral endplates.

19. An intervertebral prosthesis as claimed in claim 16, wherein said centre of curvature lies on a median plane through the centre of the first and second vertebral bodies.

20. An intervertebral prosthesis as claimed in claim 1, wherein said first and second components are formed of biocompatible material.

21. An intervertebral prosthesis as claimed in claim 20, wherein said biocompatible material is selected from the group consisting of: Titanium, Cobalt Chrome, Alumina or Zirconia Alloys, or any combination of metals or ceramic biomaterials.

22. An intervertebral prosthesis as claimed in claim 1, wherein at least one of said first and second surfaces is provided with a coating or modified surface layer.

23. (canceled)
